(19) United States
(12) Patent Application Publication
(10) Pub. No.: US 2023/0331665 A1
(43) Pub. Date: Oct. 19, 2023

(54) RHEOLOGY-MODIFYING DIFUNCTIONAL COMPOUND

(71) Applicant: COATEX, Genay (FR)

(72) Inventors: Yves MATTER, Reyrieux (FR); Denis RUHLMANN, Genay (FR); Jean-Marc SUAU, Lucenay (FR)

(73) Assignee: COATEX, Genay (FR)

(21) Appl. No.: 18/005,006

(22) PCT Filed: Jul. 26, 2021
(86) PCT No.: PCT/FR2021/000085

§ 371 Date: Jan. 10, 2023

(30) Foreign Application Priority Data
Jul. 27, 2020 (FR) FR2007895

(51) Int. Cl. C07C 265/00; A01N 25/02; A01N 47/30; A01P 1/00; C07C 263/16; C09D 5/14; C09D 7/63; C09D 7/61; C09D 105/00; C09D 17/00

(52) U.S. Cl. CPC C07C 265/00; A01N 25/02; A01N 47/30; A01P 1/00; C07C 263/16; C09D 5/14; C09D 7/63; C09D 7/61; C09D 105/00; C09D 17/001; C09D 17/008

(57) ABSTRACT

A rheology-modifying difunctional compound prepared by reacting one molar equivalent of a non-alkoxylated compound (a) and one molar equivalent of a polyethoxylated compound (b). A method for preparing the difunctional compound by reacting one molar equivalent of a non-alkoxylated compound (a) and one molar equivalent of a polyethoxylated compound (b). An aqueous composition with the difunctional compound and an additive. An aqueous formulation with the aqueous composition, an organic or mineral pigment, and an agent. A coating formulation with the aqueous formulation. A concentrated aqueous pigment pulp with the difunctional compound and coloured organic or mineral pigment. A method for controlling the viscosity of an aqueous composition.

The invention relates to a rheology-modifying difunctional compound. The invention also provides an aqueous composition comprising a difunctional compound according to the invention and a method for controlling the viscosity of an aqueous composition using the difunctional compound according to the invention.

In general, for aqueous coating compositions, and in particular for aqueous paint or varnish compositions, it is necessary to control the viscosity both for low or medium shear gradients and for high shear gradients. Indeed, during its preparation, storage, application or drying, a paint formulation is subjected to numerous stresses requiring particularly complex rheological properties.

When paint is stored, the pigment particles tend to settle by gravity. Stabilising the dispersion of these pigment particles therefore requires a paint formulation with high viscosity at very low shear gradients corresponding to the limiting velocity of the particles. Paint uptake is the amount of paint taken up by an application tool such as, for example, a paintbrush, a brush or a roller. If the tool takes up a large amount of paint when dipped into and removed from the can, it will not need to be dipped as often. Paint uptake increases as the viscosity increases. The calculation of the equivalent shear gradient is a function of the paint flow velocity for a particular thickness of paint on the tool. The paint formulation should therefore also have a high viscosity at low or medium shear gradients.

Moreover, the paint must have a high filling property so that, when applied to a substrate, a thick coat of paint is deposited at each stroke. A high filling property therefore makes it possible to obtain a thicker wet film of paint with each stroke of the tool. The paint formulation must therefore have a high viscosity at high shear gradients.

High viscosity at high shear gradients will also reduce or eliminate the risk of splattering or dripping when the paint is being applied.

Reduced viscosity at low or medium shear gradients will also result in a neat, taut appearance after the paint has been applied, particularly a single-coat paint, to a substrate which will then have a very even surface finish with no bumps or indentations. The final visual appearance of the dry coat is thus greatly improved.

Furthermore, once the paint has been applied to a surface, especially a vertical surface, it should not run. The paint formulation thus needs to have a high viscosity at low and medium shear gradients.

Lastly, once the paint has been applied to a surface, it should have a high levelling capacity. The paint formulation must then have a reduced viscosity at low and medium shear gradients.

Document EP0761780 discloses diurethane compounds that are thickening and resistant to temperature increases. Document JPH06322392 describes detergent dissolution additives that are prepared from diols or polyethers. Document EP0295031 discloses a surfactant compound for a cross-linkable isocyanate resin prepared from polyethers. Document U.S. Pat. No. 4,301,083 relates to the preparation of polyether derivatives from halides. HEUR-type compounds (hydrophobically modified ethoxylated urethane) are known as rheology-modifying agents.

However, the known HEUR-type compounds do not always make it possible to provide a satisfactory solution. In particular, the rheology-modifying compounds of the prior art do not always allow for effective viscosity control or do not always achieve a satisfactory improvement in the compromise between Stormer viscosity (measured at low or medium shear gradients and expressed in KUs) and ICI viscosity (measured at high or very high shear gradients and expressed in s⁻¹). In particular, the known rheology-modifying compounds do not always make it possible to increase the ICI viscosity/Stormer viscosity ratio.

There is therefore a need for improved rheology-modifying agents. The difunctional compound according to the invention makes it possible to provide a solution to all or part of the problems of the rheology-modifying agents in the prior art.

Thus, the invention provides a difunctional compound T prepared by reacting:
a. one molar equivalent of at least one non-alkoxylated compound (a) chosen among:
   the straight aliphatic monoisocyanate compounds (a1) comprising from 6 to 40 non-alkoxylated carbon atoms,
   the branched aliphatic monoisocyanate compounds (a2) comprising from 6 to 40 non-alkoxylated carbon atoms,
   the cycloaliphatic monoisocyanate compounds (a3) comprising from 6 to 40 non-alkoxylated carbon atoms,
   the monoaromatic monoisocyanate compounds (a4) comprising from 6 to 30 non-alkoxylated carbon atoms,
   the polyaromatic monoisocyanate compounds (a5) comprising from 10 to 80 non-alkoxylated carbon atoms,
   the straight aliphatic monohalogen compounds (a6) comprising from 6 to 40 non-alkoxylated carbon atoms,
   the branched aliphatic monohalogen compounds (a7) comprising from 6 to 40 non-alkoxylated carbon atoms,
   the cycloaliphatic monohalogen compounds (a8) comprising from 6 to 40 non-alkoxylated carbon atoms,
   the monoaromatic monohalogenoalkylene compounds (a9) comprising from 7 to non-alkoxylated carbon atoms,
   the polyaromatic monohalogenoalkylene compounds (a10) comprising from 10 to 80 non-alkoxylated carbon atoms, and
b. one molar equivalent of at least one polyethoxylated compound (b) chosen among:
   the straight aliphatic monoalcohols (b1) comprising from 6 to 40 polyethoxylated carbon atoms comprising strictly more than 100 and up to 500 oxyethylene groups,
   the branched aliphatic monoalcohols (b2) comprising from 6 to 40 polyethoxylated carbon atoms comprising from 80 to 500 oxyethylene groups,
   the cycloaliphatic monoalcohols (b3) comprising from 6 to 40 polyethoxylated carbon atoms comprising from 80 to 500 oxyethylene groups,
   the monoaromatic monoalcohols (b4) comprising from 6 to 30 polyethoxylated carbon atoms comprising strictly more than 100 and up to 500 oxyethylene groups,
   the polyaromatic monoalcohols (b5) comprising from 10 to 80 polyethoxylated carbon atoms comprising from 80 to 500 oxyethylene groups.

Advantageously according to the invention, compounds (a1) to (a5) are derived from the prior reaction of a diisocyanate compound and, respectively:
of a straight aliphatic monoalcohol comprising from 6 to 40 non-alkoxylated carbon atoms,
of a branched aliphatic monoalcohol comprising from 6 to 40 non-alkoxylated carbon atoms,
of a cycloaliphatic monoalcohol comprising from 6 to 40 non-alkoxylated carbon atoms,
of a monoaromatic monoalcohol comprising from 6 to 30 non-alkoxylated carbon atoms,
of a polyaromatic monoalcohol comprising from 10 to 80 non-alkoxylated carbon atoms.

According to the invention, the diisocyanate compounds are symmetric diisocyanate compounds or asymmetric diisocyanate compounds. The symmetric diisocyanate compounds comprise two isocyanate groups that have the same reactivity. The asymmetric diisocyanate compounds comprise two isocyanate groups that have different reactivities.

Preferably according to the invention, the diisocyanate compound is a compound in which the two isocyanate groups have different reactivities. The diisocyanate compound may be chosen among the asymmetric diisocyanate compounds, preferably IPDI. The diisocyanate compound may also be 2,6 TDI.

Generally, the diisocyanate compound may be chosen among:
certain symmetric aromatic diisocyanate compounds, preferably:
2,2′-diphenylmethylene diisocyanate (2,2′-MDI) and 4,4′-diphenylmethylene diisocyanate (4,4′-MDI);
4,4′-dibenzyl diisocyanate (4,4′-DBDI);
2,6-toluene diisocyanate (2,6-TDI);
m-xylylene diisocyanate (m-XDI);
   certain symmetric alicyclic diisocyanate compounds, preferably methylene bis(4-cyclohexylisocyanate) (H₁₂MDI);
   certain symmetric aliphatic diisocyanate compounds, preferably hexamethylene diisocyanate (HDI), pentamethylene diisocyanate (PDI);
   the asymmetric aromatic diisocyanate compounds, preferably:
2,4′-diphenylmethylene diisocyanate (2,4′-MDI);
2,4′-dibenzyl diisocyanate (2,4′-DBDI);
2,4-toluene diisocyanate (2,4-TDI);
   the asymmetric alicyclic diisocyanate compounds, preferably isophorone diisocyanate (IPDI).

Essentially according to the invention, the difunctional compound T is prepared from at least one compound (a1) to (a5) comprising an isocyanate group or from at least one compound (a6) to (a10) comprising a halogen atom and from a compound (b) capable of reacting with this isocyanate group or with this halogen atom and comprising a saturated, unsaturated, or aromatic hydrocarbon chain combined with a polyethoxylated chain.

Preferably according to the invention, this reagent compound (b) is a monohydroxyl compound.

Preferably according to the invention, the condensation of compounds (a1) to (a5) and of compound (b) is carried out in the presence of a catalyst. This catalyst can be chosen among an amine, preferably 1,8-diazabicyclo[5.4.0]undec-7-ene (DBU), a derivative of a metal chosen among Al, Bi, Sn, Hg, Pb, Mn, Zn, Zr, Ti. Traces of water may also participate in the catalysis of the reaction. As examples of metal derivatives, a derivative is preferably chosen among dibutyl bismuth dilaurate, dibutyl bismuth diacetate, dibutyl bismuth oxide, bismuth carboxylate, dibutyltin dilaurate, dibutyltin diacetate, dibutyltin oxide, a mercury derivative, a lead derivative, zinc salts, manganese salts, a compound comprising chelated zirconium, a compound comprising chelated aluminium. The preferred metal derivative is chosen among a Bi derivative, an Sn derivative and a Ti derivative.

Also preferably according to the invention, the condensation of compounds (a6) to (a10) and of compound (b) is carried out in the presence of a catalyst, in particular a base, for example a strong base, such as KOH, NaOH.

Preferably according to the invention, the reaction uses a single compound (a) or the reaction uses two or three different compounds (a).

Preferably according to the invention, the monohalogen compound is chosen among a chlorine compound, a bromine compound, an iodine compound and combinations thereof; preferably, the monohalogen compound is a bromine compound.

According to the invention, monoaromatic monohalogenoalkylene compounds (a9) are compounds comprising a single aromatic monohalogen group via an alkylene group. The halogen atom is not directly carried by the aromatic group. According to the invention, polyaromatic monohalogenoalkylene compounds (a10) are compounds comprising at least two aromatic groups of which at least one is molohalogenated via an alkylene group. The halogen atom is not directly carried by an aromatic group.

Preferably according to the invention, compound (a) is such that:
the hydrocarbon chain of compound (a1) or of compound (a6) comprises from 6 to 30 carbon atoms, preferably from 6 to 20 carbon atoms or from 8 to 16 carbon atoms; more preferentially, compound (a1) or compound (a6) is chosen among non-alkoxylated n-octanyl, non-alkoxylated n-decanyl, non-alkoxylated n-dodecanyl, non-alkoxylated n-hexadecanyl, or
the hydrocarbon chain of compound (a2) or of compound (a7) comprises from 6 to 30 carbon atoms, preferably from 6 to 20 carbon atoms or from 8 to 16 carbon atoms; more preferentially, compound (a2) or compound (a7) is chosen among non-alkoxylated ethyl hexanyl, non-alkoxylated isooctanyl, non-alkoxylated isononanyl, non-alkoxylated isodecanyl, non-alkoxylated propyl heptanyl, non-alkoxylated butyloctanyl, non-alkoxylated isododecanyl, non-alkoxylated isohexadecanyl, an alkyl group derived from a non-alkoxylated oxo alcohol, an alkyl group derived from a non-alkoxylated Guerbet alcohol, or
the hydrocarbon chain of compound (a3) or of compound (a8) comprises from 6 to 30 carbon atoms, preferably from 6 to 20 carbon atoms or from 8 to carbon atoms; more preferentially, compound (a3) or compound (a8) is chosen among non-alkoxylated ethyl-cyclohexanyl, non-alkoxylated n-nonyl-cyclohexanyl, non-alkoxylated n-dodecyl-cyclohexanyl, or
the hydrocarbon chain of compound (a4) or of compound (a9) comprises from 12 to 30 carbon atoms or from 12 to 22 carbon atoms; preferably, compound (a4) or compound (a9) is chosen among non-alkoxylated n-pentadocecyl phenyl or
the hydrocarbon chain of compound (a5) or of compound (a10) comprises from to 60 carbon atoms; preferably, compound (a5) or compound (a10) is chosen among non-alkoxylated naphtyl, non-alkoxylated distyrylphenyl, non-alkoxylated tristyrylphenyl, non-alkoxylated pentastyrylcumyl phenyl.

According to the invention, the monoalcohols are compounds comprising a single hydroxyl (OH) group that is terminal. According to the invention, the polyethoxylated monoalcohols are compounds comprising a hydrocarbon chain that comprises several oxyethylene groups and a terminal hydroxyl (OH) group. According to the invention, the polyethoxylated monoalcohols are compounds of formula R-(LO)ₙ—H in which R represents a hydrocarbon chain, n represents the number of polyethoxylations and L, identical or different, independently represents a straight alkylene group comprising 2 carbon atoms. According to the invention, the non-alkoxylated monoalcohols are compounds comprising a hydrocarbon chain and a single hydroxyl (OH) group that is terminal. According to the invention, the non-alkoxylated monoalcohols are compounds of formula R′—OH in which R′ represents a hydrocarbon chain. According to the invention, the number of carbon atoms defining monoalcohols (b1) to (b5) therefore corresponds to the number of carbon atoms in the R or R′ groups.

Preferably according to the invention, polyethoxylated monoalcohols (b1) and (b3) comprise from 105 to 400 ethoxylated groups or from 105 to 200 ethoxylated groups. Preferably according to the invention, polyethoxylated monoalcohols (b2), (b4) and (b5) comprise from 80 to 400 ethoxylated groups or from 100 to 200 ethoxylated groups.

According to the invention, the polyethoxylated compounds (b) used may comprise a number of identical or different ethoxylated groups. According to the invention, the ethoxylated groups are (—CH₂CH₂O) oxyethylene groups.

Essentially according to the invention, the compound T is a compound comprising ethoxylated groups. Preferentially according to the invention, the compound T has a degree of polyethoxylation comprised between 100 and 500 or between 100 and 502. The degree of polyethoxylation defines the number of ethoxylated groups comprised in this compound.

Preferably according to the invention, compound (b) is such that:
the hydrocarbon chain of compound (b1) comprises from 6 to 30 carbon atoms, preferably from 6 to 20 carbon atoms or from 8 to 16 carbon atoms; more preferentially, compound (b1) is chosen among polyethoxylated n-octanol, polyethoxylated n-decanol, polyethoxylated n-dodecanol, polyethoxylated n-hexadecanol, or
the hydrocarbon chain of compound (b2) comprises from 6 to 30 carbon atoms, preferably from 6 to 20 carbon atoms or from 8 to 16 carbon atoms; more preferentially, compound (b2) is chosen among polyethoxylated ethylhexanol, polyethoxylated isooctanol, polyethoxylated isononanol, polyethoxylated isodecanol, polyethoxylated propyl heptanol, polyethoxylated butyl octanol, polyethoxylated isododecanol, polyethoxylated isohexadecanol, a polyethoxylated oxo alcohol, a polyethoxylated Guerbet alcohol, or
the hydrocarbon chain of compound (b3) comprises from 6 to 30 carbon atoms, preferably from 6 to 20 carbon atoms or from 8 to 20 carbon atoms; more preferentially, compound (b3) is chosen among polyethoxylated ethylcyclohexanol, polyethoxylated n-nonyl-cyclohexanol, polyethoxylated n-dodecyl-cyclohexanol, or
the hydrocarbon chain of compound (b4) comprises from 12 to 30 carbon atoms or from 12 to 22 carbon atoms; preferably, compound (b4) is chosen among polyethoxylated n-pentadocecylphenol or
the hydrocarbon chain of compound (b5) comprises from 10 to 60 carbon atoms; preferably, compound (b5) is chosen among polyethoxylated naphthol, polyethoxylated distyrylphenol, polyethoxylated tristyrylphenol, polyethoxylated pentastyrylcumylphenol.

Essentially according to the invention, the compound T is prepared using a monoalcohol and in the absence of diol or of triol or in the absence of any compound comprising at least two hydroxyl (OH) groups.

In addition to a difunctional compound T, the invention also relates to a method for preparing this compound.

Thus, the invention provides a method for preparing a difunctional compound T by reacting:
a. one molar equivalent of at least one non-alkoxylated compound (a) chosen among:
   the straight aliphatic monoisocyanate compounds (a1) comprising from 6 to non-alkoxylated carbon atoms,
   the branched aliphatic monoisocyanate compounds (a2) comprising from 6 to non-alkoxylated carbon atoms,
   the cycloaliphatic monoisocyanate compounds (a3) comprising from 6 to non-alkoxylated carbon atoms,
   the monoaromatic monoisocyanate compounds (a4) comprising from 6 to non-alkoxylated carbon atoms,
   the polyaromatic monoisocyanate compounds (a5) comprising from 10 to 80 non-alkoxylated carbon atoms,
   the straight aliphatic monohalogen compounds (a6) comprising from 6 to non-alkoxylated carbon atoms,
   the branched aliphatic monohalogen compounds (a7) comprising from 6 to non-alkoxylated carbon atoms,
   the cycloaliphatic monohalogen compounds (a8) comprising from 6 to 40 non-alkoxylated carbon atoms,
   the monoaromatic monohalogenoalkylene compounds (a9) comprising from 7 to non-alkoxylated carbon atoms,
   the polyaromatic monohalogenoalkylene compounds (a10) comprising from to 80 non-alkoxylated carbon atoms, and
b. one molar equivalent of at least one polyethoxylated compound (b) chosen among:
   the straight aliphatic monoalcohols (b1) comprising from 6 to 40 polyethoxylated carbon atoms comprising strictly more than 100 and up to 500 oxyethylene groups,
   the branched aliphatic monoalcohols (b2) comprising from 6 to 40 polyethoxylated carbon atoms comprising from 80 to 500 oxyethylene groups,
   the cycloaliphatic monoalcohols (b3) comprising from 6 to 40 polyethoxylated carbon atoms comprising from 80 to 500 oxyethylene groups,
   the monoaromatic monoalcohols (b4) comprising from 6 to 30 polyethoxylated carbon atoms comprising strictly more than 100 and up to 500 oxyethylene groups,
   the polyaromatic monoalcohols (b5) comprising from 10 to 80 polyethoxylated carbon atoms comprising from 80 to 500 oxyethylene groups.

Preferably according to the invention for the preparation method according to the invention, the condensation of compounds (a1) to (a5) and (b) is carried out in the presence of a catalyst. More preferably, the reaction is catalysed using an amine, preferably using 1,8-diazabicyclo[5.4.0]undec-7-ene (DBU), or of at least one derivative of a metal chosen among Al, Bi, Sn, Hg, Pb, Mn, Zn, Zr, Ti. Traces of water may also participate in the catalysis of the reaction. As examples of metal derivatives, a derivative is preferably chosen among dibutyl bismuth dilaurate, dibutyl bismuth diacetalate, dibutyl bismuth oxide, bismuth carboxylate, dibutyltin dilaurate, dibutyltin diacetate, dibutyltin oxide, a mercury derivative, a lead derivative, zinc salts, manganese salts, a compound comprising chelated zirconium, a compound comprising chelated aluminium. The preferred metal derivative is chosen among a Bi derivative, an Sn derivative and a Ti derivative.

Also preferably according to the invention, the condensation of compounds (a6) to (a10) and of compound (b) is carried out in the presence of a catalyst, in particular a base, for example a strong base, such as KOH, NaOH.

Advantageously according to the invention, the condensation of compounds (a) and (b) is carried out in an organic solvent. The preferred organic solvents are solvents that are non-reactant with the isocyanate groups or with the halogen atoms of compound (a), in particular the solvents chosen among the hydrocarbon solvents (particularly C8 to Cao petroleum cuts), the aromatic solvents (particularly toluene and its derivatives) and combinations thereof. More preferably according to the invention, condensation is carried out directly with the different reagents or is carried out in toluene.

At the end of the preparation of the compound T according to the invention, a solution of the compound in an organic solvent is obtained. Such a solution can be used directly. Also according to the invention, the organic solvent can be separated and the compound T dried.

Such a compound T according to the invention, which is dried, can then be used in solid form, for example in powder or pellet form.

In addition to the difunctional compound T and a method for preparing this compound, the invention also relates to an aqueous composition comprising at least one difunctional compound T according to the invention. The invention also relates to an aqueous composition comprising at least one difunctional compound T prepared according to the preparation method according to the invention.

Advantageously, the difunctional compound according to the invention is a compound having a hydrophilic character. It can be formulated in an aqueous medium.

The aqueous composition according to the invention may also comprise at least one additive, in particular an additive chosen among:
an amphiphilic compound, in particular a surfactant compound, preferably a hydroxylated surfactant compound, for example alkyl-polyalkylene glycol, in particular alkyl-polyethylene glycol and alkyl-polypropylene glycol;
a polysaccharide derivative, for example cyclodextrin, cyclodextrin derivative, polyethers, alkyl-glucosides;
solvents, in particular coalescing solvents, and hydrotropic compounds, for example glycol, butyl glycol, butyldiglycol, mono propylene glycol, ethylene glycol, ethylenediglycol, Dowanol products with CAS number 34590-94-8), Texanol products with CAS number 25265-77-4);
anti-foaming agents, biocides.

The invention also provides an aqueous formulation that can be used in many technical fields. The aqueous formulation according to the invention comprises at least one composition according to the invention and may comprise at least one organic or mineral pigment or organic, organo-metallic or mineral particles, for example calcium carbonate, talc, kaolin, mica, silicates, silica, metal oxides, in particular titanium dioxide, iron oxides. The aqueous formulation according to the invention can also comprise at least one agent chosen among a particle-spacer agent, a dispersing agent, a stabilising steric agent, an electrostatic stabilising agent, an opacifying agent, a solvent, a coalescing agent, an anti-foaming agent, a preservative agent, a biocide, a spreading agent, a thickening agent, a film-forming copolymer and mixtures thereof.

Depending on the particular difunctional compound or the additives that it comprises, the formulation according to the invention can be used in many technical fields. Thus, the formulation according to the invention can be a coating formulation. Preferably, the formulation according to the invention is an ink formulation, an adhesive formulation, a varnish formulation, a paint formulation, for example a decorative paint or an industrial paint. Preferably, the formulation according to the invention is a paint formulation.

The invention also provides a concentrated aqueous pigment pulp comprising at least one difunctional compound T according to the invention or at least one difunctional compound T prepared according to the preparation method according to the invention and at least one coloured organic or mineral pigment.

The difunctional compound according to the invention has properties that make it possible to use it to modify or control the rheology of the medium comprising it. Thus, the invention also provides a method for controlling the viscosity of an aqueous composition.

This viscosity control method according to the invention comprises the addition of at least one difunctional compound T according to the invention to an aqueous composition. This viscosity control method may also include the addition of at least one difunctional compound T prepared according to the preparation method according to the invention.

Preferably, the viscosity control method according to the invention is carried out using an aqueous composition according to the invention. Also preferably, the viscosity control method according to the invention is carried out using an aqueous formulation according to the invention.

The particular, advantageous or preferred characteristics of the difunctional compound T according to the invention define aqueous compositions according to the invention, formulations according to the invention, pigment pulp and viscosity control methods which are also particular, advantageous or preferred.

The following examples illustrate the various aspects of the invention.

### EXAMPLES

### Example 1: Preparation of Difunctional Compounds According to the Invention

### Example 1-1: Preparation of a Compound T1 According to the Invention

In a 3 L glass reactor (container 1) equipped with mechanical stirring, a vacuum pump, a nitrogen inlet and heated by means of a double jacket in which oil circulates, 450.60 g of ethoxylated dodecanol are introduced with 140 mol of ethylene oxide (MM=6,355 Da) that is heated to 90° C. in an inert atmosphere. This product is dehydrated.

At the same time, in a 100 mL glass three-neck flask (container 2), 15.76 g of IPDI are introduced, to which 400 ppm of bismuth carboxylate catalyst is added. The medium is flushed with nitrogen, then heated to 50° C. When this temperature is reached, 9.23 g of octanol are gradually introduced.

When the injection is completed, the reaction mixture is left to stir for 15 minutes. Then, back titration is used to check that the theoretical level of NCO groups has been reached. 1 g is collected from the reaction medium to which an excess of dibutylamine (1 mol, for example) is added, which reacts with any isocyanate groups present in the medium. The unreacted dibutylamine is then dosed with hydrochloric acid (for example 1 N). The number of isocyanate groups present in the reaction medium can then be deduced.

Then, the contents of container 2 are poured into container 1. Stirring is continued for 60 minutes at 90±1° C. Then, the NCO group level is checked to ensure it is zero, indicating the end of the reaction. If this number is not zero, the reaction is continued for 15-minute periods until the reaction is completed. When the level reaches zero, the compound T1 obtained is formulated in water to which is added 1,000 ppm of a biocide (Biopol SMV Chemipol) and 1,000 ppm of an anti-foaming agent (Tego 1488 Evonik). A composition 1 is obtained consisting of 20% by mass of compound T1 according to the invention and 80% by mass of water.

### Example 1-2: Preparation of a Compound T2 According to the Invention

In a 3 L glass reactor (container 1) equipped with mechanical stirring, a vacuum pump, a nitrogen inlet and heated by means of a double jacket in which oil circulates, 448.10 g of ethoxylated dodecanol are introduced with 140 mol of ethylene oxide (MM=6,355 Da) that is heated to 90° C. in an inert atmosphere. This product is dehydrated.

At the same time, in a 100 mL glass three-neck flask (container 2), 15.67 g of IPDI are introduced, to which 400 ppm of bismuth carboxylate catalyst is added. The medium is flushed with nitrogen, then heated to 50° C. When this temperature is reached, 11.16 g of decanol are gradually introduced.

When the injection is completed, the reaction mixture is left to stir for 15 minutes. Then, back titration is used to check that the theoretical level of NCO groups has been reached.

Then, the contents of container 2 are poured into container 1. Stirring is continued for 60 minutes at 90±1° C. Then, the NCO group level is checked to ensure it is zero, indicating the end of the reaction. If this number is not zero, the reaction is continued for 15-minute periods until the reaction is completed. When the level reaches zero, the compound T2 obtained is formulated in water to which is added 1,000 ppm of a biocide (Biopol SMV Chemipol) and 1,000 ppm of an anti-foaming agent (Tego 1488 Evonik). A composition 2 is obtained consisting of 20% by mass of compound T2 according to the invention and 80% by mass of water.

### Example 1-3: Preparation of a Compound T3 According to the Invention

In a 3 L glass reactor (container 1) equipped with mechanical stirring, a vacuum pump, a nitrogen inlet and heated by means of a double jacket in which oil circulates, 453.00 g of ethoxylated dodecanol are introduced with 140 mol of ethylene oxide (MM=6,355 Da) that is heated to 90° C. in an inert atmosphere. This product is dehydrated.

At the same time, in a 100 mL glass three-neck flask (container 2), 15.85 g of IPDI are introduced, to which 400 ppm of bismuth carboxylate catalyst is added. The medium is flushed with nitrogen, then heated to 50° C. When this temperature is reached, 13.28 g of dodecanol are gradually introduced.

When the injection is completed, the reaction mixture is left to stir for 15 minutes. Then, back titration is used to check that the theoretical level of NCO groups has been reached.

Then, the contents of container 2 are poured into container 1. Stirring is continued for 60 minutes at 90±1° C. Then, the NCO group level is checked to ensure it is zero, indicating the end of the reaction. If this number is not zero, the reaction is continued for 15-minute periods until the reaction is completed. When the level reaches zero, the compound T3 obtained is formulated in water to which is added 1,000 ppm of a biocide (Biopol SMV Chemipol) and 1,000 ppm of an anti-foaming agent (Tego 1488 Evonik). A composition 3 is obtained consisting of 20% by mass of compound T3 according to the invention and 80% by mass of water.

### Example 1-4: Preparation of a Compound T4 According to the Invention

In a 3 L glass reactor (container 1) equipped with mechanical stirring, a vacuum pump, a nitrogen inlet and heated by means of a double jacket in which oil circulates, 449.80 g of ethoxylated dodecanol are introduced with 140 mol of ethylene oxide (MM=6,355 Da) that is heated to 90° C. in an inert atmosphere. This product is dehydrated.

At the same time, in a 100 mL glass three-neck flask (container 2), 15.73 g of IPDI are introduced, to which 400 ppm of bismuth carboxylate catalyst is added. The medium is flushed with nitrogen, then heated to 50° C. When this temperature is reached, 17.16 g of hexadecanol are gradually introduced.

When the injection is completed, the reaction mixture is left to stir for 15 minutes. Then, back titration is used to check that the theoretical level of NCO groups has been reached.

Then, the contents of container 2 are poured into container 1. Stirring is continued for 60 minutes at 90±1° C. Then, the NCO group level is checked to ensure it is zero, indicating the end of the reaction. If this number is not zero, the reaction is continued for 15-minute periods until the reaction is completed. When the level reaches zero, the compound T4 is formulated using a surfactant compound such as ethoxylated alcohol (ethoxylated octanol with ten ethylene oxide equivalents) in water to which is added 1,000 ppm of a biocide (Biopol SMV Chemipol) and 1,000 ppm of an anti-foaming agent (Tego 1488 Evonik). A composition 4 is obtained consisting of 20% by mass of compound T4 according to the invention, 10% of surfactant compound and 70% by mass of water.

### Example 1-5: Preparation of a Compound T5 According to the Invention

In a 3 L glass reactor (container 1) equipped with mechanical stirring, a vacuum pump, a nitrogen inlet and heated by means of a double jacket in which oil circulates, 452.50 g of ethoxylated dodecanol are introduced with 140 mol of ethylene oxide (MM=6,355 Da) that is heated to 90° C. in an inert atmosphere. This product is dehydrated.

At the same time, in a 100 mL glass three-neck flask (container 2), 15.83 g of IPDI are introduced, to which 400 ppm of bismuth carboxylate catalyst is added. The medium is flushed with nitrogen, then heated to 50° C. When this temperature is reached, 19.08 g of 4-dodecylcyclohexanol are gradually introduced.

When the injection is completed, the reaction mixture is left to stir for 15 minutes. Then, back titration is used to check that the theoretical level of NCO groups has been reached.

Then, the contents of container 2 are poured into container 1. Stirring is continued for 60 minutes at 90±1° C. Then, the NCO group level is checked to ensure it is zero, indicating the end of the reaction. If this number is not zero, the reaction is continued for 15-minute periods until the reaction is completed. When the level reaches zero, the compound T5 is formulated using a surfactant compound such as ethoxylated alcohol (ethoxylated octanol with ten ethylene oxide equivalents) in water to which is added 1,000 ppm of a biocide (Biopol SMV Chemipol) and 1,000 ppm of an anti-foaming agent (Tego 1488 Evonik). A composition 5 is obtained consisting of 20% by mass of compound T5 according to the invention, 10% of surfactant compound and 70% by mass of water.

### Example 2: Preparation of Paint Formulations According to the Invention

Paint formulations F1 to F5 according to the invention are prepared respectively from aqueous compositions 1 to 5 of difunctional compounds T1 to T5 according to the invention. All of the ingredients and proportions (% by mass) used are listed in Table 1.

TABLE 1

| Ingredients | Amount (g) |
|---|---|
| water | 99.7 |
| dispersing agent (Coadis BR3 Coatex) | 3.9 |
| biocide (Acticide MBS Thor) | 1.3 |
| anti-foaming agent (Airex 901W Evonik) | 1.31 |
| NH₄OH (28%) | 0.6 |
| TiO₂ pigment (RHD2 Huntsman) | 122.2 |
| CaCO₃ pigment (Omyacoat 850 OG Omya) | 84.6 |
| binding agent (Acronal S790 Basf) | 270.7 |
| monopropylene glycol | 6.5 |
| solvent (Texanol Eastman) | 6.5 |
| anti-foaming agent (Tego 825 Evonik) | 1.0 |
| aqueous composition 1 according to the | 28.7 |
| invention | |
| added water | q.s.p. 650 g total |

### Example 3: Characterisation of Paint Formulations According to the Invention

For the paint formulations according to the invention, the Brookfield viscosity, measured at 25° C. and at 10 rpm and 100 rpm (μ_{Bk10} and μ_{Bk100} in mPa·s) was determined 24 hours after their preparation using a Brookfield DV-1 viscometer with RVT spindles. The

TABLE 2

| Formulation | Compound | μ_{BK10} | μ_{BK100} |
|---|---|---|---|
| F1 | T1 | 1,020 | 590 |
| F2 | T2 | 1,380 | 810 |
| F3 | T3 | 1,530 | 940 |
| F4 | T4 | 3,250 | 1,720 |
| F5 | T5 | 3,150 | 1,570 |

properties of the paint formulations are listed in Table 2.

The difunctional compounds according to the invention are highly effective in obtaining excellent low and medium shear gradient viscosities for paint compositions.

### Example 4: Characterisation of Paint Formulations According to the Invention

For the paint formulations according to the invention, the Cone Plan viscosity or ICI viscosity, measured at high shear gradient (μI in mPa·s) was determined 24 hours after their preparation and at room temperature, using a Cone & Plate Research Equipment London (REL) viscometer having a measuring range of 0 to 5 poise, and the Stormer viscosity, measured at medium shear gradient (μS in Krebs Units or KUs), was determined using the reference module of a Brookfield KU-2 viscometer. The properties of the paint formulations are listed in Table 3.

TABLE 3

| Formulation | Compound | μI | μs | μI/μS |
|---|---|---|---|---|
| F1 | T1 | 150 | 72 | 2.0 |
| F2 | T2 | 150 | 78 | 1.9 |
| F3 | T3 | 140 | 82 | 1.7 |
| F4 | T4 | 250 | 96 | 2.6 |
| F5 | T5 | 250 | 94 | 2.6 |

The difunctional compounds according to the invention make it possible to prepare paint formulations with particularly well-controlled viscosities. In particular, the μ_{I} viscosity is particularly high and the μ_{I}/μₛ ratio is therefore excellent. The compounds according to the invention allow for an excellent compromise between high shear gradient viscosity and low shear gradient viscosity.

1. A difunctional compound T, obtained by reacting:
one molar equivalent of at least one non-alkoxylated compound (a) selected from the group consisting of:
straight aliphatic monoisocyanate compounds (a1) comprising from 6 to 40 non-alkoxylated carbon atoms,
branched aliphatic monoisocyanate compounds (a2) comprising from 6 to 40 non-alkoxylated carbon atoms,
cycloaliphatic monoisocyanate compounds (a3) comprising from 6 to non-alkoxylated carbon atoms,
monoaromatic monoisocyanate compounds (a4) comprising from 6 to non-alkoxylated carbon atoms,
polyaromatic monoisocyanate compounds (a5) comprising from 10 to 80 non-alkoxylated carbon atoms,
straight aliphatic monohalogen compounds (a6) comprising from 6 to non-alkoxylated carbon atoms,
branched aliphatic monohalogen compounds (a7) comprising from 6 to 40 non-alkoxylated carbon atoms,
cycloaliphatic monohalogen compounds (a8) comprising from 6 to non-alkoxylated carbon atoms,
monoaromatic monohalogenoalkylene compounds (a9) comprising from 7 to 30 non-alkoxylated carbon atoms, and
polyaromatic monohalogenoalkylene compounds (a10) comprising from 10 to 80 non-alkoxylated carbon atoms; and
one molar equivalent of at least one polyethoxylated compound (b) selected from the group consisting of:
straight aliphatic monoalcohols (b1) comprising from 6 to 40 polyethoxylated carbon atoms comprising strictly more than 100 and up to 500 oxyethylene groups,
branched aliphatic monoalcohols (b2) comprising from 6 to 40 polyethoxylated carbon atoms comprising from 80 to 500 oxyethylene groups,
cycloaliphatic monoalcohols (b3) comprising from 6 to 40 polyethoxylated carbon atoms comprising from 80 to 500 oxyethylene groups,
monoaromatic monoalcohols (b4) comprising from 6 to 30 polyethoxylated carbon atoms comprising strictly more than 100 and up to 500 oxyethylene groups, and
polyaromatic monoalcohols (b5) comprising from 10 to 80 polyethoxylated carbon atoms comprising from 80 to 500 oxyethylene groups.

2. The difunctional compound T according to claim 1, wherein the reaction is with a single compound (a) or two or three different compounds (a).

3. The difunctional compound T according to claim 1, wherein the monohalogen compound is at least one selected from the group consisting of a chlorine compound, a bromine compound, an iodine compound and combinations thereof.

4. The difunctional compound T according to claim 1, wherein:
a degree of polyalkoxylation is from 100 and 500; or
the polyethoxylated monoalcohols (b1) and (b3) comprise from 105 to 400 ethoxylated groups or from 105 to 200 ethoxylated groups; or
polyethoxylated monoalcohols (b2), (b4) and (b5) comprise from 80 to 400 ethoxylated groups or from 100 to 200 ethoxylated groups; or
the polyethoxylated compound (b) comprises a number of ethoxylated groups that is identical or different.

5. The difunctional compound T according to claim 1, wherein compound (a) is:
a hydrocarbon chain of compound (a1) or of compound (a6) comprising from 6 to 30 carbon atoms;
a hydrocarbon chain of compound (a2) or of compound (a7) comprising from 6 to 30 carbon atoms;
a hydrocarbon chain of compound (a3) or of compound (a8) comprising from 6 to 30 carbon atoms;
a hydrocarbon chain of compound (a4) or of compound (a9) comprising from 12 to 30 carbon atoms or from 12 to 22 carbon atoms; and
a hydrocarbon chain of compound (a5) or of compound (a10) comprising from 10 to 60 carbon atoms.

6. The difunctional compound T according to claim 1, wherein compound (b) is:
a hydrocarbon chain of compound (b1) comprising from 6 to 30 carbon atoms;
a hydrocarbon chain of compound (b2) comprising from 6 to 30 carbon atoms;
a hydrocarbon chain of compound (b3) comprising from 6 to 30 carbon atoms;
a hydrocarbon chain of compound (b4) comprising from 12 to 30 carbon atoms or from 12 to 22 carbon atoms;
a hydrocarbon chain of compound (b5) comprising from 10 to 60 carbon atoms.

7. A method for preparing a difunctional compound T, comprising reacting:
one molar equivalent of at least one non-alkoxylated compound (a) selected from the group consisting of:
straight aliphatic monoisocyanate compounds (a1) comprising from 6 to 40 non-alkoxylated carbon atoms,
branched aliphatic monoisocyanate compounds (a2) comprising from 6 to 40 non-alkoxylated carbon atoms,
cycloaliphatic monoisocyanate compounds (a3) comprising from 6 to non-alkoxylated carbon atoms,
monoaromatic monoisocyanate compounds (a4) comprising from 6 to non-alkoxylated carbon atoms,
polyaromatic monoisocyanate compounds (a5) comprising from 10 to 80 non-alkoxylated carbon atoms,
straight aliphatic monohalogen compounds (a6) comprising from 6 to non-alkoxylated carbon atoms,
branched aliphatic monohalogen compounds (a7) comprising from 6 to 40 non-alkoxylated carbon atoms,
cycloaliphatic monohalogen compounds (a8) comprising from 6 to non-alkoxylated carbon atoms,
monoaromatic monohalogenoalkylene compounds (a9) comprising from
7 to 30 non-alkoxylated carbon atoms, and
polyaromatic monohalogenoalkylene compounds (a10) comprising from 10 to 80 non-alkoxylated carbon atoms; and
one molar equivalent of at least one polyethoxylated compound (b) selected from the group consisting of:
straight aliphatic monoalcohols (b1) comprising from 6 to 40 polyethoxylated carbon atoms comprising strictly more than 100 and up to 500 oxyethylene groups,
branched aliphatic monoalcohols (b2) comprising from 6 to 40 polyethoxylated carbon atoms comprising from 80 to 500 oxyethylene groups,
cycloaliphatic monoalcohols (b3) comprising from 6 to 40 polyethoxylated carbon atoms comprising from 80 to 500 oxyethylene groups,
monoaromatic monoalcohols (b4) comprising from 6 to 30 polyethoxylated carbon atoms comprising strictly more than 100 and up to 500 oxyethylene groups, and
polyaromatic monoalcohols (b5) comprising from 10 to 80 polyethoxylated carbon atoms comprising from 80 to 500 oxyethylene groups.

8. The method according to claim 7, wherein the reaction employs a single compound (a).

9. An aqueous compositions comprising:
at least one a difunctional compound T according to claim 1, and optionally
at least one additive selected from the group consisting of:
an amphiphilic compound;
a polysaccharide derivative;
solvents; and
anti-foaming agents or biocides.

10. An aqueous formulation comprising:
the composition according to claim 9; and optionally
at least one organic or mineral pigment or organic, organo-metallic or mineral particle selected from the group consisting of calcium carbonate, talc, kaolin, mica, silicates, silica, metal oxides, in particular titanium dioxide, iron oxides; and optionally
at least one agent selected from the group consisting of a particle-spacer agent, a dispersing agent, a stabilising steric agent, an electrostatic stabilising agent, an opacifying agent, a solvent, a coalescing agent, an anti-foaming agent, a preservative agent, a biocide, a spreading agent, a thickening agent, a film-forming copolymer and mixtures thereof.

11. A coating formulation, comprising the aqueous formulation according to claim 10.

12. A concentrated aqueous pigment pulp comprising:
the difunctional compound T according to claim 1 and a coloured organic or mineral pigment.

13. A method for controlling the viscosity of an aqueous composition, comprising:
adding the difunctional compound T according to claim 1 to the aqueous solution.

14. The method according to claim 13, wherein the aqueous composition a coating formulation.

15. The difunctional compound T according to claim 3, wherein the monohalogen compound is bromine.

16. The difunctional compound T according to claim 5, wherein the hydrocarbon chain of compound (a1) or of compound (a6) has from 6 to 20 carbon atoms or from 8 to 16 carbon atoms and is at least one selected from the group consisting of a non-alkoxylated n-octanyl, non-alkoxylated n-decanyl, non-alkoxylated n-dodecanyl, and non-alkoxylated n-hexadecanyl;
the hydrocarbon chain of compound (a2) or of compound (a7) has from 6 to 20 carbon atoms or from 8 to 16 carbon atoms and is at least one selected from the group consisting of non-alkoxylated ethyl hexanyl, non-alkoxylated isooctanyl, non-alkoxylated isononanyl, non-alkoxylated isodecanyl, non-alkoxylated propyl heptanyl, non-alkoxylated butyloctanyl, non-alkoxylated isododecanyl, non-alkoxylated isohexadecanyl, an alkyl group derived from a non-alkoxylated oxo alcohol, and an alkyl group derived from a non-alkoxylated Guerbet alcohol;
the hydrocarbon chain of compound (a3) or of compound (a8) has from 6 to 20 carbon atoms or from 8 to 20 carbon atoms and is at least one selected from the group consisting of non-alkoxylated ethyl-cyclohexanyl, non-alkoxylated n-nonyl-cyclohexanyl, and non-alkoxylated n-dodecyl-cyclohexanyl;
the compound (a4) or compound (a9) is a non-alkoxylated n-pentadocecyl phenyl; and
the compound (a5) or compound (a10) is at least one selected from the group consisting of non-alkoxylated naphtyl, non-alkoxylated distyrylphenyl, non-alkoxylated tristyrylphenyl, non-alkoxylated pentastyrylcumyl phenyl.

17. The difunctional compound T according to claim 6, wherein the hydrocarbon chain of compound (b1) comprises from 6 to 20 carbon atoms or from 8 to 16 carbon atoms and is at least one selected from the group consisting of polyethoxylated n-octanol, polyethoxylated n-decanol, polyethoxylated n-decanol, polyethoxylated n-dodecanol, and polyethoxylated n-hexandecanol;
the hydrocarbon chain of compound (b2) comprises from 6 to 20 carbon atoms or from 8 to 16 carbon atoms and is at least one selected from the group consisting of polyethoxylated ethylhexanol, polyethoxylated isooctanol, polyethoxylated isononanol, polyethoxylated isodecanol, polyethoxylated propyl heptanol, polyethoxylated butyl octanol, polyethoxylated isododecanol, polyethoxylated isohexadecanol, a polyethoxylated oxo alcohol, and a polyethoxylated Guerbet alcohol;
the hydrocarbon chain of compound (b3) comprises from 6 to 20 carbon atoms or from 8 to 20 carbon atoms and is at least one selected from the group consisting of polyethoxylated ethylcyclohexanol, polyethoxylated n-nonyl-cyclohexanol, and polyethyoxylated n-dodecyl-cyclohexanol;
the hydrocarbon chain of compound (b4) is a polyethoxylated n-pentadocecylphenol; and
the hydrocarbon chain of compound (b5) is at least one selected from the group consisting of polyethoxylated naphthol, polyethoxylated distyrylphenol, polyethoxylated tristyrylphenol, and polyethoxylated pentastyrylcumylphenol.

18. The aqueous composition of claim 9, wherein the amphiphilic compound is at least one surfactant compound selected from the group consisting of alkyl-polyalkylene glycol, alkyl-polyethylene glycol, and alkyl-polypropylene glycol;
the polysaccharide derivative is at least one selected from the group consisting of cyclodextrin, cyclodextrin derivative, polyethers, and alkyl-glucosides; and
the solvent is at least one selected from the group consisting of glycol, butyl glycol, butyldiglycol, mono propylene glycol, ethylene glycol, and ethylenediglycol,

19. The coating formulation of claim 11, wherein the coating formulation is an ink formulation, a varnish formulation, an adhesive formulation, a paint formulation for decorative paint, and a paint formulation for an industrial paint.
